(19) United States
(12) Patent Application Publication
(10) Pub. No.: US 2006/0172022 A1
(43) Pub. Date: Aug. 3, 2006

(54) Skin treating salve composition

(76) Inventor: Sharon Szanzer, Brooklyn, NY (US)

Correspondence Address: OSTROLENK FABER GERB & SOFFEN, 1180 AVENUE OF THE AMERICAS, NEW YORK, NY, 100368403, US

(21) Appl. No.: 11/066,177

(22) Filed: Jan. 31, 2005

(51) Int. Cl. A61K 36/48; A61K 36/898; A61K 36/63; A61K 36/61

(52) U.S. Cl. 424/757; 424/769; 424/776

(57) ABSTRACT

A salve composition and method for topically treating a variety of deleterious skin conditions and for maintaining the wellness of skin comprises, as active ingredients, 100% pure hydrogenated vegetable oil and cocoa butter and a variety of enhancers, such as olive oil, jojoba oil and primrose oil. The basic composition can be enhanced with the addition of one or more of SPF as a ultraviolet agent, lavender oil for maintaining skin conditions, grapeseed oil and buffered vitamin C powder as a gentle skin cleanser and hydrogenated vegetable and cocoa butter oil or hydrogenated vegetable and cocoa butter oil and beeswax as a lip protectant; and tea tree oil to help fight eczema, diaper rash, Tea Tree Oil and Zinc6 for more irritated diaper rash or rectal or vaginal irritation, Bee Pollen for certain cases of psoriasis and combination of Eucalyptus Oil, Peppermint Oil and Cayenne Pepper to help muscle and joint pain, minor skin irritations and insect bites. The salve with tea tree oil and lavender may also be utilized as a mild insect repellent with no harmful side effects. The original formula helps prevent or minimize stretch marks during and after pregnancy.

### BACKGROUND OF THE INVENTION

This invention relates generally to a salve composition having medicinal properties in the treatment of skin conditions and disorders. More particularly, the present invention pertains to a medicinal salve composition with two primary active ingredients, namely, a mixture of pure hydrogenated vegetable oil and pure cocoa butter, for topically treating dry irritable skin and to generally help maintain and restore skin to suppleness and wholesome conditions.

### SUMMARY OF THE INVENTION

It is an object of the present invention to provide a salve composition having medicinal properties in the treatment of skin disorders and the maintenance of wellness of skin.

The foregoing and other objects of the invention are realized with medicinal salve composition having two primary active ingredients, namely pure hydrogenated vegetable oil and pure cocoa butter, supplemented by various enhancers. In accordance with the preferred embodiment, a basic composition includes an admixture of pure hydrogenated vegetable oil, pure cocoa butter, olive oil, jojoba oil and primrose oil. Preferably, the vegetable oil may be soybean oil. The basic composition can be enhanced with tea tree oil for the treatment of eczema, psoriasis, cracked or dry feet with or without fungus, diaper rash, insect bites, mild allergic skin reactions and mild acne. Another variation includes lavender oil to maintain a healthy complexion and general overall well conditioned skin. A natural sunscreen may be included, for example, zinc oxide or 50% composition of SPF 40.

Other features and advantages of the present invention will become apparent from the following description of the invention which refers to the accompanying drawings.

### DETAILED DESCRIPTION OF PREFERRED EMBODIMENTS

The Basic Formulation

The basic formulation includes preferably nearly 100% pure hydrogenated vegetable oil and preferably pure cocoa butter, as the primary active ingredients, with optional enhancers. In accordance with one embodiment of the present invention that has been reduced to practice, the salve composition is a substantially homogeneous mixture comprising the following ingredients in the following preferred proportions:

about 1.75 cups of pure hydrogenated vegetable oil;

about 4 oz. of pure cocoa butter;

about 4 ml. olive oil;

about 4 ml. jojoba oil; and

about 4 ml. primrose oil.

When utilizing the term “about”, the instant inventor perceives that for the preferred composition, content may vary by about ±10%. When utilizing the term “cup”, eight ounces of volume are meant.

The above ingredients are processed as follows. First, the hydrogenated vegetable oil is set aside in a stainless steel bowl.

The cocoa butter is slowly placed in a stainless steel pot which is on a low flame, until the cocoa butter is entirely melted. When still on the flame, the olive oil and jojoba oil are added to the cocoa butter, while stirring gently until the mixture is thoroughly blended. Thereafter, the cocoa butter mixture is blended into the hydrogenated vegetable oil mixture by whipping, gradually increasing the whipping speed during the course of about twelve minutes.

The thus prepared and blended mixture is slowly poured into a glass bottle and is thereafter ready for use as described further on herein.

Although the basic composition described above was indicated as a preferred mixture proportion, the mixture can be prepared with a less tolerant specification as noted below:

about 0.125-8 cups of pure hydrogenated vegetable oil;

about 0.125-32 ounces of pure cocoa butter;

about 0.005-80 ml. olive oil;

about 0.005-80 ml. jojoba oil; and

about 0.005-80 ml. primrose oil.

Basic Formulation with Tea Tree Oil

The above described formulation can be further enhanced with the addition of tea tree oil which has been found to be especially suited for in the treatment of eczema, psoriasis, cracked or dry feet with or without fungus, diaper rash, insect bites, mild allergic skin reactions and mild acne. In more severe allergic reactions it was recommended to call physician for amount of antihistamine to be administered followed by application of cream with tea tree oil to be applied liberally every 3-4 hours. The tea tree oil based formulation has also been formulated to help any skin irritation that has itchiness associated with it. It is believed that the tea tree oil acts as an antibacterial agent to help fight infection or fungus. However, the formulation including the tea tree oil can also be utilized as a simple moisturizer and skin conditioner to help maintain an acne-free complexion due to the unique properties of the tea tree oil in combination with other essential oils and cocoa butter.

Preferably, the original formula is modified by adding about 3.5 ml. of tea tree oil, with the concentration being increased in dependence on the severity of the eczema or skin irritation and also based on the age of a person, for which the concentration of the tea tree oil is increased gradually.

The tea tree oil is added after the primrose oil is added to the cocoa butter mixture. The blending of the mixture is continued on the flame for another sixty seconds. Immediately thereafter the cocoa butter mixture is taken and blended into the pure hydrogenated vegetable oil mixture by whipping, gradually increasing the speed of whipping for about twelve minutes. The preferred concentration which is about 3.5 ml. of the tea tree oil can be varied so that it is included in the amount of from 0.005-80 ml. of the tea tree oil.

The Basic Formulation with Lavender Oil

The basic formulation can be blended with lavender oil to help maintain a healthy complexion and general overall well conditioned skin, and to benefit from the lavender oil's antiseptic qualities. The formulation with lavender oil should be avoided with individuals who have skin allergies. The formulation is prepared in the same manner and with the same ingredients as in the original formula, except that 3.5 ml. of lavender oil is added after the primrose oil is added to the cocoa butter mixture. The mixture is continuously blended on a flame for another sixty seconds. Thereafter the cocoa butter mixture is blended into the hydrogenated vegetable oil mixture by whipping, gradually increasing the speed for a period of about twelve minutes.

The lavender oil, while preferably including the indicated amount of 3.5 ml., can be included in different amounts for example 0.005-80 ml. of lavender oil.

Formulations Including a Natural Sunscreen

The above described formulation can include a sunscreen. In this instance, the same ingredients are used for the individually described creams, but each has a 50% ration of SPF 40 added to cream, giving an SPF factor of 20. All three of these formulas can be used either as a moisturizer, skin conditioner, skin protectant or sunscreen.

The formulations including the 50% ratio of SPF 40 can be added to any of the aforementioned formulations, i.e., the original basic formula, the original with tea tree oil or with lavender oil after one has melted the cocoa butter. The formulation is kept on the flame and the other oils are continually blended into the mixture except the hydrogenated oils. The blending continues for about sixty seconds. Thereafter, the cocoa butter mixture is taken and blended into the hydrogenated vegetable oil mixture by whipping, gradually increasing the speed for about twelve minutes. The SPF 40 is then added to the mixture to provide a 50/50 ratio of total concentration; i.e., 8 oz. of cream mixture to 8 oz. of SPF 40 mixture.

Gently Acting Cleanser

The invention also comprises preparing the formulation to act as a gentle skin cleanser. Preferably, the formulation includes about:

1.5 cup of pure hydrogenated vegetable oil;

4 oz. cocoa butter;

4 ml. olive oil;

4 ml. jojoba oil;

4 ml. primrose oil;

10 ml. grapeseed oil; and

10 ml. buffered vitamin C powder.

The aforementioned ingredients are combined in the same way as the original formulation, except that the grapeseed oil and the buffered vitamin C powder are added after the primrose oil is added to the cocoa butter mixture. Blending continues on a small flame for about 30 seconds and then the mixture is immediately removed from the flame and blended into the hydrogenated vegetable oil mixture for another twelve minutes.

The cleanser formulation ingredient proportion can be varied as indicated below:

0.125-8 cup of hydrogenated vegetable oil;

0.125-32 oz. cocoa butter;

0.005-80 ml. olive oil;

0.005-80 ml. jojoba oil;

0.005-80 ml. primrose oil;

0.005-1000 ml. grapeseed oil; and

0.005-1000 ml. buffered vitamin C powder.

Lip Protectant Formulation

The present ingredients can be substantially used in a slightly different formulation to serve as a lip protectant. The formulation includes about:

0.625 cups hydrogenated vegetable oil;

4 oz. cocoa butter;

4 ml. olive oil;

4 ml. jojoba oil;

4 ml. primrose oil.

The foregoing ingredients are combined with one another and formulated utilizing a process and method described with respect to the formulation which includes the sunscreen.

Lip Protectant Formulation with Beeswax

The lip protectant can be made with added beeswax for subjects who prefer the scent and feel of beeswax. The formulation includes about:

0.625 cups hydrogenated vegetable oil;

4 oz. cocoa butter;

4 oz. beeswax;

4 ml. olive oil;

4 ml. jojoba oil;

4 ml. primrose oil.

The foregoing ingredients are combined with one another and formulated utilizing a process and method described with respect to the formulation which includes the sunscreen. The proportions of the ingredients can be varied as indicated below:

0.005-8 cups hydrogenated vegetable oil;

0.125-32 oz. cocoa butter;

0.125-32 oz. beeswax;

0.005-80 ml. olive oil;

0.005-80 ml. jojoba oil;

0.005-80 ml. primrose oil.

The proportions of the ingredients can be varied as indicated below:

0.005-8 cups hydrogenated vegetable oil;

0.125-32 oz. cocoa butter;

0.005-80 ml. olive oil;

0.005-80 ml. jojoba oil; and

0.005-80 ml. primrose oil.

Experiment Results

Basic Formulation

As noted above, the basic formulation was evolved to maintain normal healthy skin and to protect skin from outside elements.

The basic formula was tested on six subjects with skin dryness ranging from mild dryness to severe dryness on their legs and feet. Improvement began to show overnight. For those with dryness noted on their feet, the cream was applied liberally to the feet, massaged in and covered with clean cotton socks. The subjects observed in the morning a great deal of improvement in the skin condition of their feet. At that time another application of the formulation was applied and again in the evening before retiring while covering the feet with cotton socks or the like. By the second day of this process, in some subjects, the dryness was gone. In subjects with very dry skin conditions, it took about 3-4 days for the feet to be soft or for their cracked skin to be healed entirely. In the subjects who had extreme dryness, it was suggested initial use overnight should be applied liberally and then wrapped with plastic wrap and removed in the morning. This was only done for extreme cases of dryness.

The above mentioned subjects were recommended to utilize the formulation with the tea tree oil to help fight any fungus or infection that might be present in their feet.

There are no harmful side effects whatsoever in applying the cream with the tea tree oil. It is especially beneficial if there are any open abrasions, sores, fungus, eczema, psoriasis or infection present. The tea tree oil acts as an antibacterial to help fight infection or fungus. In the subjects with severe skin dryness due to either lymphedema, eczema or psoriasis, upon application of the cream through gentle massage motions, there was an immediate feeling of the skin being softened, thus rendering the skin less itchy and irritated. In the severe cases, applying the cream at least three times daily and at night using a cotton covering the skin showed significant improvements from day to day. The skin became able to shed dead skin cells easily and allowed the new healthy skin to appear.

This process was observed to take between one to two weeks, depending on how diligently the subject applied the cream. It was recommended to the subjects that their clothes and linens be only washed in fragrance free soaps and that they be double rinsed. The soap that the subjects utilize on their bodies should be of the mildest nature and also preferably fragrance free. It was also suggested and recommended that, for children with eczema, their baths be prepared with a small amount of baking soda in lieu of any soap until the skin clears of any open wounds. The baking soda acts as a natural gentle cleanser and helps neutralize the water. As soon as the subjects came out of their the bath or shower, the skin was to be immediately gently patted dry and massaged liberally with the formulation with tea tree oil, while the skin felt still somewhat damp. In the morning, the cream was again to be massaged in the skin. As soon as the cream was applied, it helped immediately to relieve the feeling of tight itchy irritated skin. If the subject felt itchy or irritated at any time, it was suggested that the cream be applied again. There are no harmful effects of using the cream as often as the subject felt necessary to feel or obtain relief.

In another case study using four subjects who had severe dry skin on their hands, there was significant improvement upon application of the basic cream of the instant invention. If the skin had any cuts or abrasions or open wounds, it was recommended that they used cream with tea tree oil. If the skin had just skin irritation, it was recommended that they use the basic formula. Upon application of the formulation, subjects felt immediate relief. Their skin felt less itchy and relieved. The formulation was applied originally at least three times daily if there were any open wounds. At night, the hands, just as with the feet, were to be covered with a clean, cotton covering depending on the degree of skin dryness. The experience produced hands that were healed during a period of between 1 and 5 days. In the subjects who had extreme dryness, it was suggested initial use overnight should be applied liberally and then wrapped with plastic wrap and removed in the morning. This was only done for extreme cases of dryness.

In another case study using six subjects who had chapped skin on face and hands, here was significant improvement upon application of the basic cream of the instant invention. The ages of the subjects were 1 month to 10 years old. Since the skin was irritated primarily due to the temperature elements, it was recommended that the basic cream be applied prior to facing the elements outside and again before retiring to sleep. Upon application of the formulation, subjects felt immediate relief. The experience produced skin that was healed depending upon the severity of chappness in 1 to 3 days.

In another case study using four subjects with sunburn, there was gradual improvement, depending upon the condition of the sunburn. The subjects were 16-35 year old individuals. It was first recommended if it was a first degree burn, to cool the burn down by applying cool compresses to the affected area for 10 minutes, remove for 10 minutes, apply for another 20 minutes, remove for 10 minutes, and if the burn is still felt, reapply the compress for another 20 minutes. When actual burning sensation on skin decreased after application of cold compresses, basic formulation of cream was to be applied liberally, but very gently. The basic formulation of cream was to be applied a minimum of three times daily. If the burning sensation resumed, more applications of cold compresses was to be applied accordingly. It was also advised that all burns are to be treated as more serious if accompanied by other injuries or medical problems. If subject felt a decreased blood pressure, medical attention was advised immediately. In the subjects with a simple first degree burn, with no other medical complications, the healing process began after 24 hours with the help of the basic formulation of the cream. There are not any irritants that would hurt or aggravate already damaged skin

In another case study using ten subjects who used the cream for just normal everyday skin conditioning to help fight the signs of aging, the observed results were literally remarkable. First, the subjects were recommended to utilize the basic formula, or the basic formula with tea tree oil or lavender, whichever one they preferred. The recommendation was to use this formulation to remove eye makeup or any other type of makeup. If the subjects preferred, they were also able to use the gentle acting cleanser. The subjects then showered to gently clean their face with the remainder of cream applied to their face. If the subject preferred to deep clean their pores thoroughly, but gently, it was recommended first to apply gentle acting cleanser, rinse off with baking soda solution of 2 teaspoons baking soda to 8 ounces of distilled water. Rinse off and pat skin dry gently and apply cream (original formulation or tea tree formulation or lavender formulation as preferred) liberally. After the shower, they were to reapply the cream to the face to seal in the moisture and nutrients from the cream in their skin. The lavender or tea tree oil formula, depending on subjects' preference, was to be applied if there was any skin irritation due to any slight acne or pimples due to the antiseptic qualities found in the lavender, or the antibacterial and antiseptic qualities in the tea tree oil

In another case study using infants in the age range from seven days old through 24 months old who used the cream for infant acne or infant dry skin, the observed results were literally remarkable. The formula given to the infants was the basic formulation. The recommendation was to apply the cream two times daily by gentle massaging motion; once in the morning when the baby woke up and once before retiring. In either case, one time should be after giving a bath; then massage the cream in. The infant acne cleared up within 2-3 days. The dry skin began clearing up and healing within a 24 hour period. It was recommended to maintain continued usage of the cream twice daily even after the condition cleared up, to prevent any recurrence of any skin irritation. There were found to be no harmful side effects whatsoever in applying the cream.

In another case study using five infants and five children in an age range of three months through 10 years of age with diaper rash or other vaginal irritation; the cream with tea tree oil was applied to the diaper or vaginal area. The rash cleared up overnight. It was recommended that the cream be applied for at least three more applications to prevent reoccurrence. If the rash did not respond to the original application, it was suggested that the cream be combined with a 30% zinc oxide based formulation of Desitin™ ointment or tea tree oil and Zinc 6 as an alternative.

In another case using five infants and five children with diaper rash or vaginal or rectal irritation of age range 1 month-10 years old—cream with tea tree oil was applied to diaper or vaginal/rectal area. In the mild cases the rash cleared up overnight. It was recommended the cream be applied with at least 3 more applications to make sure the rash did not reoccur. The tea tree oil acted as an antibacterial agent to help fight fungus or infection. If the rash was not responding to cream upon initial application overnight, or if the rash looked more sever than normal diaper area irritation it is recommenced that the cream with Tea Tree Oil and Zinc 6 be applied for at least 3-5 days. Cream was to be applied liberally and often as necessary, especially after each diaper change or urinary or bowel movement. Area was first to be cleaned with a gentle cleanser or hypoallergenic baby wipe and then apply the cream. There are no harmful side effects from the cream. It was suggested to ask a pediatrician how long to use the cream for once the rash healed.

Basic Formulation with Tea Tree Oil and Zinc 6

The above described formulation can be further enhanced with the addition of Tea Tree Oil in combination with Zinc 6. The Tea Tree Oil and Zinc 6 based formulation has also been formulated to help irritated diaper rash, or irritated vaginal or rectal irritation which is more severe than simple diaper rash irritation or simple vaginal or rectal irritation. The addition of Zinc 6 helps the irritated area by forming a protective moisture barrier on the skin giving extra healing properties in combination to the other essential oils and cocoa butter.

Preferably, the original formulation is modified by adding 3.5 ml of tea tree oil and 490 mg of Zinc 6, with the concentration of Zinc 6 being increased in dependence on the severity of the rash or rectal/vaginal irritation, for which the concentration of the Zinc 6 is increased gradually.

The Tea Tree Oil and Zinc 6 are added after the primrose oil is added to the cocoa butter mixture. The blending of the mixture is continued on the flame for another sixty seconds. Immediately thereafter, the cocoa butter mixture is taken and blended into the pure hydrogenated vegetable oil mixture by whipping, gradually increasing the speed of whipping for about twelve minutes. The preferred concentration which is about 3.5 ml of the Tea Tree Oil can be varied so that it is included in the amount of from 0.005-80 ml of the Tea Tree Oil, the preferred concentration which is about 490 mg of Zinc 6 can be varied so that it is included in the amount of from 1 mg-4900 mg of Zinc 6.

In another case study using four pregnant women the cream with the basic formulation was applied once or twice daily on the abdomen region. The cream acted as a natural belly balm to help moisturize and calm itchy bellies. Also the cream due to its unique properties helped prevent or minimize stretch marks during and after pregnancy.

Basic Formulation with Eucalyptus Oil, Peppermint Oil and Cayenne Pepper

In another case using five adults for sore aches and pains in muscles and joints the cream with eucalyptus oil, peppermint oil and cayenne pepper was applied 3 to 4 times daily and massaged gently into painful area. Subjects felt immediate relief. When pain reappeared subjects reapplied the cream to find relief.

The above described formulation can be further enhanced with the addition of Eucalyptus Oil in combination with Peppermint Oil and Cayenne Pepper. This formulation has been specially formulated to help provide temporary relief of minor aches and pains in muscles and joints.

Rubbed into the skin, the combination of Eucalyptus Oil, Peppermint Oil and Cayenne Pepper in combination with basic formulation helps stimulate blood flow and give a feeling of warmth providing temporary relief of minor aches and pains in muscles and joints. Cream is to be applied liberally to painful area or areas and massaged into the skin. Repeat 3-4 times daily.

This formulation should never be given to infants. If given to children only after consulting a pediatrician and only once or twice daily. If given to pregnant woman only after consulting physician. This formulation is never to applied near the airway-nose or mouth due to Eucalyptus Oil.

Preferably, the original formula is modified by adding 5 ml of Eucalyptus Oil, 5 ml Peppermint Oil and 5 ml Cayenne Pepper with the concentration of the essential oils and cayenne pepper being increased or decreased in dependence of the severity of aches and pains in muscles and joints and also with age of subject.

The Eucalyptus Oil, Peppermint Oil and Cayenne Pepper are added after the primrose oil is added to the cocoa butter mixture. The blending of the mixture is continued on the flame for another sixty seconds.

Thereafter, the cocoa butter mixture is taken and blended into the pure hydrogenated vegetable oil mixture by whipping, gradually increasing the speed of whipping for about twelve minutes. The preferred concentration which is about 5 ml of Eucalyptus Oil, 5 ml Peppermint Oil and 5 ml Cayenne Pepper can be varied so that it is included in the amount from 0.005-5000 ml of Eucalyptus Oil, 0.005-5000 ml Peppermint Oil and 0.005-5000 ml Cayenne Pepper.

Basic Formulation with Bee Pollen

This formula was developed for subjects with psoriasis who were not responding to the formulation with Tea Tree Oil. It was also suggested to the subjects to consult a nutritionist to see how much acidophilus and flaxseed oil to be added to their diet daily. The subjects who were using the Tea Tree Oil formulation were also advised to consult a nutritionist to see how much acidophilus and flaxseed oil to be added to their diet daily. The subjects were to apply the Basic Formulation with Bee Pollen or Tea Tree Oil at least 3 times daily, or any time their skin felt itchy or irritated. Skin relief was felt immediately.

The Bee Pollen is added after the primrose oil is added to the cocoa butter mixture. The blending of the mixture is continued on the flame for another sixty seconds. Thereafter, the cocoa butter mixture is taken and blended into the pure hydrogenated vegetable oil mixture by whipping, gradually increasing the speed of whipping for about twelve minutes. The preferred concentration which is about 10 ml of Bee Pollen can be varied so that it is included in the amount of from 0.005-5000 ml of Bee Pollen.

It was also suggested that if the subject felt necessary to deep clean their skin, that a simple mixture of 2 teaspoons baking soda to 8 ounces distilled water be placed in a peri bottle, shaken up and used as a deep cleanser one to 5 times weekly. Immediately when finished to pat dry face to put on cream liberally to not only smooth and protect the skin but also help restore the skin suppleness. It also serves to increase the moisture and also helps to improve the appearance of dry skin. In this situation, the cream plays a dual role, one as a cleanser and the other as a skin conditioning and protecting moisturizing emollient. In the subjects with acne or simple outbreaks of pimples or pimple it was suggested to use baking soda mixture on skin every night, rinsing immediately, gently pat face and apply cream to face liberally. With all the subjects, the response was that the cream helped rejuvenate their damaged, irritated skin by making their skin less vulnerable to outside elements as well as by helping maintain healthy skin, and making fine wrinkle lines on the skin less noticeable.

The aforementioned formulations have been found to comprise highly potent moisturizers and skin conditions. They are all made with natural ingredients and do not include any artificial fragrances, dyes or parabens. They constitute excellent natural moisturizers and emollients that not only sooth and protect the skin but also help restore the skin suppleness, increase moisturization and improve the appearance of dry skin.

The unique blending of the ingredients thus forms them into a construct and overall formulation whose benefits far exceed the benefits of the individual ingredients if applied alone. Immediate infusion of the cream nutrients absorbs into the skin easily at normal body temperatures of 98.6° F.

The formulation penetrates the skin leaving a protective barrier against outside elements. Continuous use of the cream helps repair damaged skin and conditions the skin by strengthening the skin structure making it able to fight outside elements that normally might harm skin, e.g., airborne pollutants.

Each ingredient in the cream has its unique properties to help heal, repair and soften damaged skin. The unique properties of the cream give it multiple functions ranging from eliminating skin irritation to gentle nourishing and protecting a baby's delicate skin. Due to the unique properties of the cream, in some cases the cream may play the dual role of both a cleanser and skin conditioner, protecting and moisturizing emollient. The cream can be applied to all parts of the body, from the scalp down to the soles of the feet. It can be used on infants as well as adults without any harmful steroids or chemicals which can irritate the skin or weaken the immune system.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A salve composition for topically treating dry or damaged skin and to maintain skin healthy, said salve composition substantially comprising a homogeneous admixture of:
about 100% pure hydrogenated vegetable oil;
pure cocoa butter; and
the balance comprising enhancers.

2. The salve composition of claim 1, wherein the composition includes ingredients in the proportion of:
about 0.125-8 cups of pure hydrogenated vegetable oil;
about 0.125-32 oz. of pure cocoa butter;
about 0.005-80 ml. olive oil;
about 0.005-80 ml. jojoba oil; and
about 0.005-80 ml. primrose oil.

3. The salve composition of claim 2, wherein said composition proportionally comprises:
about 1.75 cups of pure hydrogenated vegetable oil;
about 4 oz. of pure cocoa butter;
about 4 ml. olive oil;
about 4 ml. jojoba oil; and
about 4 ml. primrose oil.

4. The salve composition of claim 1, including tea tree oil.

5. The salve composition of claim 4, including tea tree oil in an amount of about 0.005-80 ml.

6. The salve composition of claim 5, in which the tea tree oil is included in an amount of about 3.5 ml.

7. The salve composition of claim 1, including lavender oil in an amount of 0.005-80 ml.

8. The salve composition of claim 7, wherein the lavender oil is included in an amount of 3.5 ml.

9. The salve composition of claim 1, further including a sunscreen.

10. The salve composition of claim 9, wherein the sunscreen is zinc oxide.

11. The salve composition of claim 1, wherein the hydrogenated vegetable oil comprises soybean oil.

12. A skin cleansing composition including a plurality of ingredients in an amount of about:
0.125-8 cup of hydrogenated vegetable oil;
0.125-32 oz. cocoa butter;
0.005-80 ml. olive oil;
0.005-80 ml. jojoba oil;
0.005-80 ml. primrose oil;
0.005-1000 ml. grapeseed oil; and
0.005-1000 ml. buffered vitamin C powder.

13. The skin cleansing composition of claim 12, wherein the ingredients are included in a proportion of about:
1.5 cups of hydrogenated vegetable oil;
4 oz. cocoa butter;
4 ml. olive oil;
4 ml. jojoba oil;
4 ml. primrose oil;
10 ml. grapeseed oil; and
10 ml. buffered vitamin C powder.

14. A lip protectant composition comprising:
0.005-8 cups hydrogenated vegetable oil;
0.125-32 oz. cocoa butter;
0.005-80 ml. olive oil;
0.005-80 ml. jojoba oil; and
0.005-80 ml. primrose oil.

15. The lip protectant composition of claim 14, wherein the ingredients are included in a proportion of about:
0.625 cups hydrogenated vegetable oil;
4 oz. cocoa butter;
2 ml. olive oil;
4 ml. jojoba oil; and
4 ml. primrose oil.

16. The lip protectant composition of claim 14, wherein the ingredients include 4 oz. beeswax.

17. The salve composition of claim 4, including Zinc 6.

18. The salve composition of claim 1, including one or more of eucalyptus oil, peppermint oil and cayenne pepper.

19. The salve composition of claim 1, including bee pollen.

20. A method of preparing a skin maintaining and repairing cream, the method comprising topically applying to skin requiring repair, an effective amount of the composition of claim 1.
